(19) United States
(12) Patent Application Publication
(10) Pub. No.: US 2019/0256550 A1
(43) Pub. Date: Aug. 22, 2019

(54) SYSTEMS AND METHODS FOR GENERATING BIOLOGICAL MATERIAL

(71) Applicant: Raymond C. Kurzweil, Sudbury, MA (US)

(72) Inventor: Raymond C. Kurzweil, Sudbury, MA (US)

(73) Assignee: Kurzweil Technologies, Inc., Newton, MA (US)

(21) Appl. No.: 16/400,267

(22) Filed: May 1, 2019

Related U.S. Application Data
(63) Continuation of application No. 11/424,022, filed on Jun. 14, 2006.
(60) Provisional application No. 60/692,327, filed on Jun. 20, 2005.

(51) Int. Cl. C07K 1/00; C07K 1/04; G16B 50/00

(52) U.S. Cl. CPC C07K 1/00; G16B 50/00; C07K 1/045

(57) ABSTRACT

The invention relates to systems and methods for synthesizing biological material.

### CROSS REFERENCE TO RELATED APPLICATION

This application claims the benefit of the filing date of U.S. Provisional Application No. 60/692,327, which was filed on Jun. 20, 2005, the contents of which (including all appendices) are hereby incorporated by reference to this description.

### BACKGROUND

The invention relates to systems and methods for synthesizing biological material.

Methods of manipulating biological systems and processes through the use of cell-free assays and nanotechnology have developed rapidly in recent years. For example, cell-free systems for directing protein synthesis, RNA transcription, and RNA splicing are known and more efficient and robust systems are evolving, such those that include the use of very small sample volumes and that use nanotechnology. Nanotechnology has been applied to the manipulation of cells and cellular processes, including cell sorting based on the type, size, or function of a cell. Micro-fabricated fluidic channels have been developed for sizing and sorting DNA molecules. Photonic pressure has been used to transport cells over the length of defined fluidic channels. Bio-chips have been developed which have the ability to operate with extremely small sample volumes (on the order of nanoliters) and to perform analyses at much higher rates than can be achieved by traditional methods. Many of the existing bio-chip and microfluidic technologies use electrical, mechanical or forces to perform switching within the microfluidic channels. Certain optical-based technologies describe the use of lasers to define an optical path having an intensity gradient sufficient to propel the particles along a path but sufficiently weak enough that the particles are not trapped in an axial direction. Other lasers can interrogate particles to identify predetermined phenotypical characteristics, and upon recognition of a particular phenotype, can deflect the particles in along a different specified path. As the field of nanotechnology grows and the understanding of the mechanics of biological processes evolves, the two fields will continue to merge.

### SUMMARY

In some embodiments, a system includes a computer configured to execute instructions for synthesizing biological material and an assembly unit electrically connected to the computer, the assembly unit being configured to synthesize the biological material based on the instructions executed by the computer.

Embodiments can include one or more of the following.

The system can include an insertion unit. The system can include a repository unit. The assembly unit further comprises one or more of an input channel and an output channel. The system can include separate device for a user to communicate wirelessly with the computer. The computer can include one or more of a memory unit, software, and a database. The database can include one or more of DNA sequence, RNA sequence and polypeptide sequence information. The repository unit can include one or more different types of monomeric biological components. The monomeric biological components can include one or more of a nucleotide, amino acid, and tRNA. Each tRNA can be attached to an amino acid.

The assembly unit can include one or more of a polymerase or a ribosome. The assembly unit can include a robot that mimics the activity of a polymerase or a ribosome. The biological material can be a nucleic acid or polypeptide. The biological material can be an RNA.

The insertion unit can be attached to the assembly unit. The system can be coated with a biocompatible material.

In some embodiments, a computer configured to execute instructions for synthesizing biological material, a central unit responsive to execution of the instructions to control an assembly unit, and an assembly unit electrically connected to the central unit, the assembly unit being configured to synthesize the biological material based on the instructions executed by the computer.

Embodiments can include one or more of the following.

The system can include a repository unit. The assembly unit can include one or more of an input channel and an output channel. The computer can be separate from the central unit and the assembly unit. The computer can reside outside of the cell and the central unit and the assembly unit reside inside the cell. The computer can reside within the central unit, and the central unit and the assembly unit can reside inside the cell. The system can include a separate device for a user to communicate wirelessly with the computer. The computer can include one or more of a transmitter, software, and a database. The central unit can include one or more of a memory, a receiver, an engine, and an antenna. The database can include one or more of DNA sequence, RNA sequence and polypeptide sequence information.

The system can include a repository unit, and the repository unit comprises one or more different types of monomeric biological components. The monomeric biological components can include one or more of a nucleotide, amino acid, and tRNA. Each tRNA can be attached to an amino acid. The assembly unit can include one or more of a polymerase or a ribosome. The assembly unit can include a robot that mimics the activity of a polymerase or a ribosome. The biological material can be a nucleic acid or polypeptide. The biological material can include an RNA. The system can be coated with a biocompatible material.

In some embodiments, a method includes synthesizing a biological material and introducing the biological material into a cell. The biological material is synthesized by a system that includes a computer configured to execute instructions for synthesizing biological material and an assembly unit electrically connected to the computer, the assembly unit being configured to synthesize the biological material based on the instructions executed by the computer.

Embodiments can include one or more of the following.

The system further can include an insertion unit and a repository unit. The system further can include one or more of an input channel and an output channel on the assembly unit. The step of synthesizing the biological material can be initiated by a signal from a user operating a device separated from the system, wherein the user uses the device to send a signal to a receiver located in the computer of the system.

In some embodiments, a method includes synthesizing a biological material and introducing the biological material into a cell. The biological material can be synthesized by a system that includes a computer configured to execute instructions for synthesizing biological material, a central unit responsive to execution of the instructions to control an assembly unit, and an assembly unit electrically connected to the computer, the assembly unit being configured to synthesize the biological material based on the instructions executed by the computer.

Embodiments can include one or more of the following.

The system can include a repository unit. The method can also include, prior to the synthesizing step, of putting at least the central unit and the assembly unit of the system inside the cell. The system further can include one or more of an input channel and an output channel on the assembly unit. Putting the central unit and the assembly unit of the system into the cell can include electroporation, microinj ection, or a lipophilic carrier. Synthesizing the biological material can be initiated by a signal from a user operating a device separated from the system, wherein the user uses the device to send a signal to a receiver located in the central unit of the system. The computer can reside inside the central unit.

In some embodiments, a cell comprising a system includes a central unit responsive to instructions to control an assembly unit and an assembly unit electrically connected to the central unit, the assembly unit being configured to synthesize biological material based on the instructions. The instructions are executed by a computer.

Embodiments can include one or more of the following.

The computer can reside inside the central unit. The computer can reside outside the central unit and outside the cell. The system can include a repository unit attached to the assembly unit. The assembly unit can include one or more of an input channel and an output channel on the assembly unit. The computer can include one or more of a transmitter, software, and a database. The central unit can include one or more of a memory, a receiver, an engine, and an antenna. The repository unit can include one or more different types of monomeric biological components. The monomeric biological components can include one or more of a nucleotide, amino acid, and tRNA. Each tRNA can be attached to an amino acid. The cell can originate from a mammal. The cell can originate from a human, mouse, rat, monkey, dog, cat, or rabbit. The cell can be in a tissue of a human.

In some embodiments, a method of treating a human includes administering a system to the human. The system includes a central unit responsive to instructions to control an assembly unit and an assembly unit electrically connected to the central unit, the assembly unit being configured to synthesize biological material based on the instructions. The instructions are executed by a computer.

Embodiments can include one or more of the following.

The computer can reside inside the central unit. The computer can reside outside the central unit and outside the cell. The system can include a repository unit attached to the assembly unit. The assembly unit can include one or more of an input channel and an output channel. The computer can include one or more of a transmitter, software, and a database. The central unit can include one or more of a memory, a receiver, an engine, and an antenna. The database can include one or more of DNA sequence information, RNA sequence information, and polypeptide sequence information. The repository unit can include one or more different types of monomeric biological components. The monomeric biological components can include one or more of a nucleotide, amino acid, and tRNA. Each tRNA is attached to an amino acid. The human can have a cancer, a tissue disorder, or a disorder of the nervous system. The system can be administered by tissue graft, microprojectile technology, or by a lipophilic carrier.

In some embodiments, a method of treating a human includes administering a cell comprising. The system includes a central unit responsive to instructions to control an assembly unit and an assembly unit electrically connected to the central unit, the assembly unit being configured to synthesize biological material based on the instructions. The instructions are generated by a computer.

Embodiments can include one or more of the following.

The computer can reside inside the central unit. The computer can reside outside the central unit and outside the human. The system includes a repository unit attached to the assembly unit. The assembly unit of the system can include one or more of an input channel and an output channel on the assembly unit. The computer can include one or more of a transmitter, software, and a database. The central unit can include one or more of a memory, a receiver, an engine and an antenna. The database can include DNA sequence information, RNA sequence information, and polypeptide sequence information. The repository unit can include one or more different types of monomeric biological components. The monomeric biological components can include one or more of a nucleotide, amino acid, and tRNA. Each tRNA can be attached to an amino acid. The human can have a cancer, a tissue disorder, or a disorder of the nervous system. The cell can be administered by tissue graft.

The details of one or more embodiments of the invention are set forth in the accompanying drawings and the description below. Other features, objects, and advantages of the invention will be apparent from the description and drawings, and from the claims.

### DESCRIPTION OF DRAWINGS

FIG. 1 is a block diagram of a biological material synthesizing system such as a system residing outside of a cell.

FIG. 2 is a block diagram of a database.

FIG. 3 is a block diagram of a biological material synthesizing system, such as a system including components that can reside inside a cell.

FIG. 4 is flow chart of a process for generating biological material using the system of FIG. 2.

### DETAILED DESCRIPTION

Described are systems and methods for synthesizing biological materials.

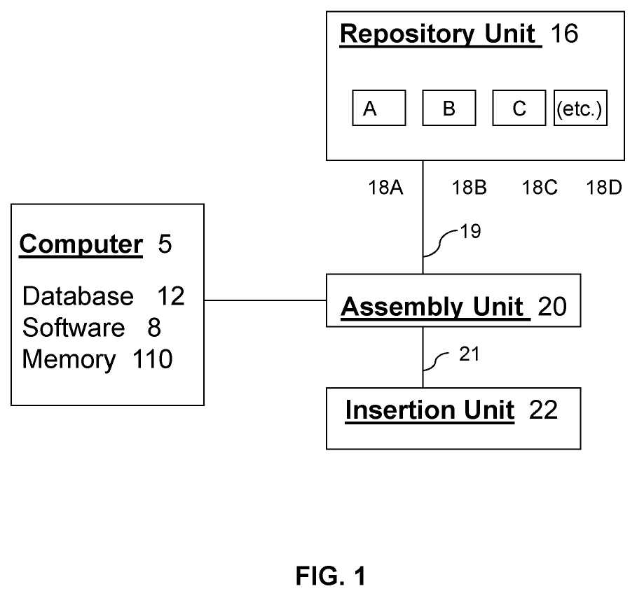

Referring to FIG. 1, the systems include at least a computer 5 and an assembly unit 20. The computer 5 is configured to generate instructions for the synthesis of biological material, and includes, for example, a database 12, software 8, and memory 110. The assembly unit 20 is electronically connected to the computer 5 and is configured to synthesize the biological material based on the instructions received from the computer. The assembly unit 20 stores machinery for synthesizing the biological material and can be located inside or outside of a cell. When the assembly unit is located outside of the cell, the assembly unit can be physically attached to the computer, and also to a repository unit 16 containing one or more different types of monomeric biological components. The repository unit 16 can be attached to the assembly unit 20 at the site of an input channel on the assembly unit 20. The assembly unit 20 uses the biological components stored in the repository unit for the synthesis of the biological material. The assembly unit 20 can also be attached to an insertion unit 22, such as at the site of an output channel on the assembly unit. Synthesized biological material can pass from the assembly unit 20 into the cell through the insertion unit 22. When the assembly unit 20 is located inside the cell, the assembly unit can be attached to a central unit 10, which is also located within the cell. The central unit can include the computer 5, or the central unit can be separated from the computer, and the computer can be stored, for example, outside the cell. The central unit can include a memory 110, receiver 14, engine 108 and antenna 102. A computer 5, located outside the cell, can include a database 12, software 8, and a transmitter 6 for transmitting instructions regarding biological synthesis from the computer to the central unit. The synthesized biological material can pass from the assembly unit directly into the cell, such as through an output channel. The biological materials synthesized by the systems featured herein can be multimeric molecules, such as nucleic acids (e.g., DNA or RNA) and polypeptides. A system that synthesizes biological material or a cell containing such a system can be used to treat a human who has a disorder, such as cancer or a neurological disorder.

Systems for Synthesizing Biological Material.

In general the computer 5 provides instructions to the assembly unit 20 indicating how to generate biological material. The assembly unit 20 generates the biological material using monomeric components (e.g., monomeric components 18a-18d) stored in the repository unit 16. In some embodiments, the system operates outside the cell, and further includes an insertion unit 22. When the system functions outside of the cell, the biological material synthesized within the assembly unit passes through the insertion unit 22 and into the cell. The insertion unit resembles microinjection apparatus known in the art. For example, the insertion unit can include an injection pipet with an external diameter of about 1 micrometer (e.g., about 0.6 micrometer, about 0.8 micrometer, about 1.0 micrometer, about 1.2 micrometer, about 1.4 micrometer) and tubing connecting the assembly unit and the injection pipet. The tubing can have an external diameter of about 60-70 micrometers (e.g., 62, 64, 66, 68 micrometers). An insertion unit includes a hollow portion that can hold biological material generated by the assembly unit. The assembly unit can be in fluid communication with the insertion unit such that biological material can flow from the assembly unit to the insertion unit. The insertion unit also includes a tip with an opening disposed at the tip. The tip is configured to pierce the membrane of cell without permanently damaging the cell. The insertion unit inserts the biological material stored in the hollow portion into the cell by flowing the biological material through the opening in the tip.

Still referring to FIG. 1, in order to generate the biological material the computer 5 communicates information with the assembly unit 20. In general, the computer 5 includes software 8 that provides instructions to the assembly unit 20. The software 8 includes a user interface that allows a user to select the type of biological material to be generated by the assembly unit 20. Based on the type of biological material the user selects to generate, the software 8 interfaces with a database 12 to determine an appropriate set of instructions to send to the assembly unit 20.

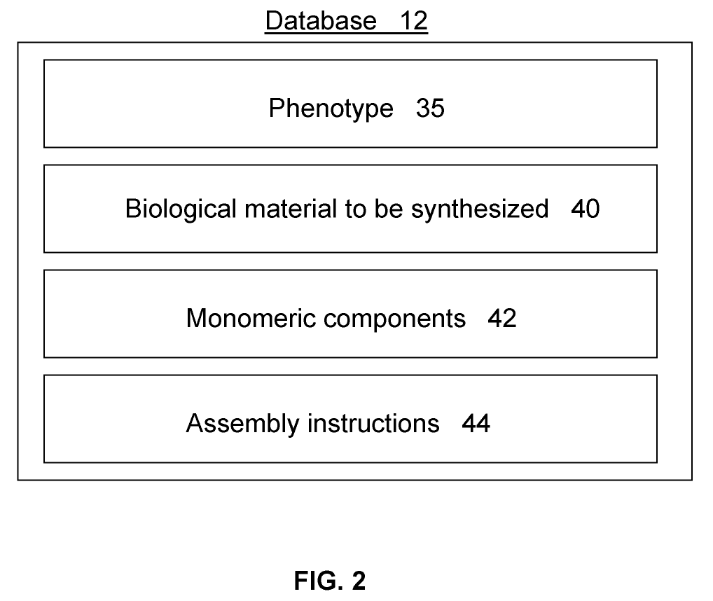

For example, as shown in FIG. 2, the database 12 can store information regarding cell phenotype 35, the type or unit of biological material to be synthesized 40, the monomeric components 42 of the biological material, and the assembly instructions 44 for the material. As depicted in FIG. 2 the database includes a table with fields representing the stored data, however other types of databases such as relational databases, or flat file systems, etc. could be used. The computer can maintain the information of a human's genetic code, altered to replace deleterious information (e.g., deleterious mutations) with benign or beneficial information. By this approach, cells that are genetically programmed to synthesize a misfunctioning or nonfunctioning polypeptide, or programmed not to synthesize a particular essential polypeptide, can be manipulated to express the properly functioning polypeptide by execution of the program by the computer and using data stored in the database.

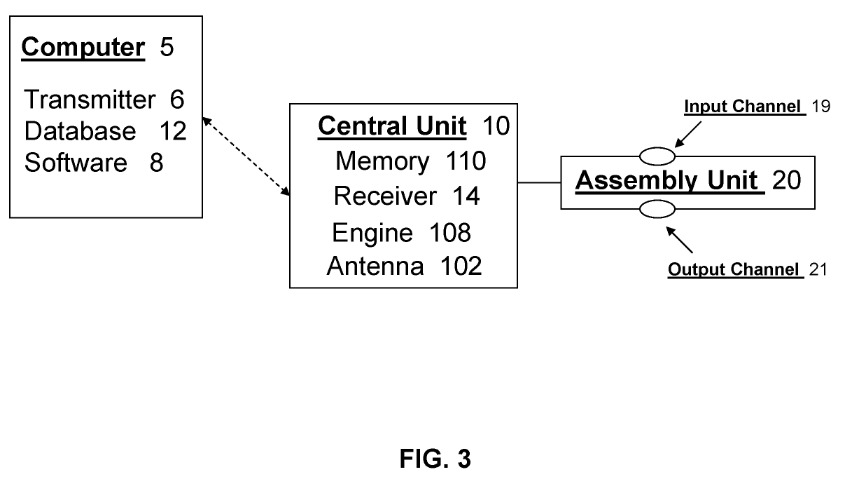

Referring to FIG. 3, an assembly unit 20 can include an input channel 19 and an output channel 21. An assembly unit located inside a cell may or may not be attached to a repository. The assembly unit 20 can harvest the biological building blocks (e.g., nucleotides, amino acids) directly from the cell cytoplasm and through the input channel 19. Synthesized biological material is released from the assembly unit and into the cell through the output channel 21.

The biological material synthesizing system can be programmed such that the assembly unit 20 receives instructions for synthesizing one particular type of biological material (e.g., nucleic acid, polypeptide). Alternatively, the assembly unit can receive instructions for synthesizing more than one type of biological material. For example, the computer can be programmed such that the assembly unit 20 receives instructions for synthesizing all the polypeptides necessary to support cell function. The computer can be programmed and re-programmed by a user using a device located outside of the cell. The device can be a wireless device, such as a second computer or a remote control device.

A repository unit can include, for example, nucleotides, such as deoxyribonucleotides for assembling deoxyribonucleic acid (DNA), or ribonucleotides for assembling ribonucleic acid (RNA), or amino acids for assembling polypeptides. DNA is a polymer of deoxyribonucleotide subunits and RNA is a polymer of ribonucleotide subunits. A nucleotide is a nitrogenous base (e.g., a purine, a pyrimidine), a sugar (e.g., a ribose, a deoxyribose), and one or more phosphate units. Table 1 lists exemplary nucleotides and amino acids that can be included in the repository unit. The repository unit can include a stock of amino acids attached to tRNA. Ribosomes within the assembly unit, or machinery that mimics a ribosome, can use the stock of tRNA to assemble a polypeptide by a mechanism similar to that which occurs in vivo.

TABLE 1
Exemplary monomeric components of the repository unit of a system.

| Component Class | Monomeric Component |
|---|---|
| Deoxynucleotides | Deoxyadenosine 5′-triphosphate |
| | Deoxyguanosine 5′-triphosphate |
| | Deoxycytodine 5′-triphosphate |
| | Deoxythymidine 5′-triphosphate |
| Ribonucleotides | adenosine 5′-triphosphate |
| | guanosine 5′-triphosphate |
| | cytodine 5′-triphosphate |
| | uridine 5′-triphosphate |
| Amino Acids | Alanine |
| | Asparagine |
| | Cysteine |
| | Aspartic acid |
| | Glutamic acid |
| | Phenylalanine |
| | Glycine |
| | Histidine |
| | Isoleucine |
| | Lysine |
| | Leucine |
| | Methionine |
| | Proline |
| | Glutamine |
| | Arginine |
| | Serine |
| | Threonine |
| | Valine |
| | Tryptophan |
| | Tyrosine |

The repository unit 16 can also include carbohydrates for attachment to the polypeptides. Alternatively, necessary carbohydrates can be attached to the polypeptide by the endogenous cellular machinery after the polypeptide passes through the assembly unit output channel.

The assembly unit 20 can include machinery for synthesizing biological material. The assembly unit 20 can include enzymes (e.g., RNA polymerases, ribosomes) to facilitate RNA and polypeptide synthesis. Alternatively, or in addition, the assembly unit 20 can include machinery (e.g., manmade machinery) that mimics the endogenous cellular machinery.

The repository unit 16 and assembly unit 20 can be fluid-filled with salts and buffer to provide an environment that mimics the interior of a cell. Such an environment facilitates the integrity of the molecular structures, including secondary and tertiary polypeptide structures formed as the amino acids are linked together in the assembly unit. The salts can include, for example, potassium, magnesium, calcium, zinc, ammonium or sodium salts, while suitable buffers include MES, Tris, HEPES, MMT and the like. The fluid can also include serum, such as bovine fetal serum, to provide additional components to support assembly of the biological materials. Other suitable components include reducing agents, such as dithiothreitol (DTT), chelating agents such as EDTA, and polymers such as polyethylene glycols (PEGs). The fluid within a system can also contain antibacterial and antifungal agents to prevent contamination. Appropriate antibacterial agents include but are not limited to streptomycin and penicillin, and fungizone is an example of an appropriate antifungal agent.

A technique for assembling the biological material can be programmed into the computer 5, and the programmable interface and the internal operating circuitry and/or the signal processor, which may be one or more of a microprocessor or nanoprocessor, can be configured to allow adjustable and/or selectable operational configurations of the device to operate in the desired feedback mode or modes. The computer 5 can communicate with the assembly unit directly (FIG. 1) or can communicate wirelessly with a central unit 10 (FIG. 3). The computer and assembly unit can operate outside of the cell (see, e.g., FIG. 1), or the system, or parts of the system, can be introduced into the interior of the cell, where it can assemble the biological material and distribute the material directly into the interior of the cell (see, e.g., FIG. 3).

Referring to FIG. 3, a wireless system can include a computer 5, a central unit 10, and an assembly unit 20. The computer can reside inside the cell (such as inside a central unit 10), or the computer 5 can reside outside the cell. A computer residing outside the cell can send instructions to the central unit that resides inside the cell. Due to the limited size of the central unit, some or all information processing can occur in a processor located on the computer 5. For example, the database 12 can be stored on the computer 5 and a reduced set of data or instructions (e.g., and executable instruction set) can be wirelessly transmitted from a transmitter 6 on computer 5 to a receiver 14 on the central unit 10. The central unit 10 stores the received instructions or computer executable code in a memory 110. During operation, the central unit 10 executes the instructions stored on the memory 110 and sends instructions to the assembly unit 20 for generation of biological material.

The processor can be a microprocessor, a nanoprocessor, or a set of micro-engines. In general, a microprocessor is a computer processor on a microchip. A nanoprocessor can be a processor having limited memory for executing a reduced set of instructions.

In general, a processor can be implemented in digital electronic circuitry, in computer hardware, firmware, software, or in combinations of these. The processor described herein can be implemented as a computer program product, e.g., a computer program tangibly embodied in an information carrier, e.g., in a machine-readable storage device or in a propagated signal, for execution by, or to control the operation of, a data processing apparatus, e.g., a processing device, a computer, or multiple computers. A computer program can be written in any form of programming language, including compiled, assembled, or interpreted languages, and it can be deployed in any form, including as a stand-alone program or as a module, component, subroutine, or other unit suitable for use in a computing environment. A computer program can be deployed to be executed on one computer or on multiple computers at one site or distributed across multiple sites and interconnected by a communication network.

The system can be coated in a material that is biocompatible, such as a coating formed from polyurethane, or an amorphous titanium nitride. A material is biocompatible if it can come in contact with at least one part of the body without causing significant health hazards.

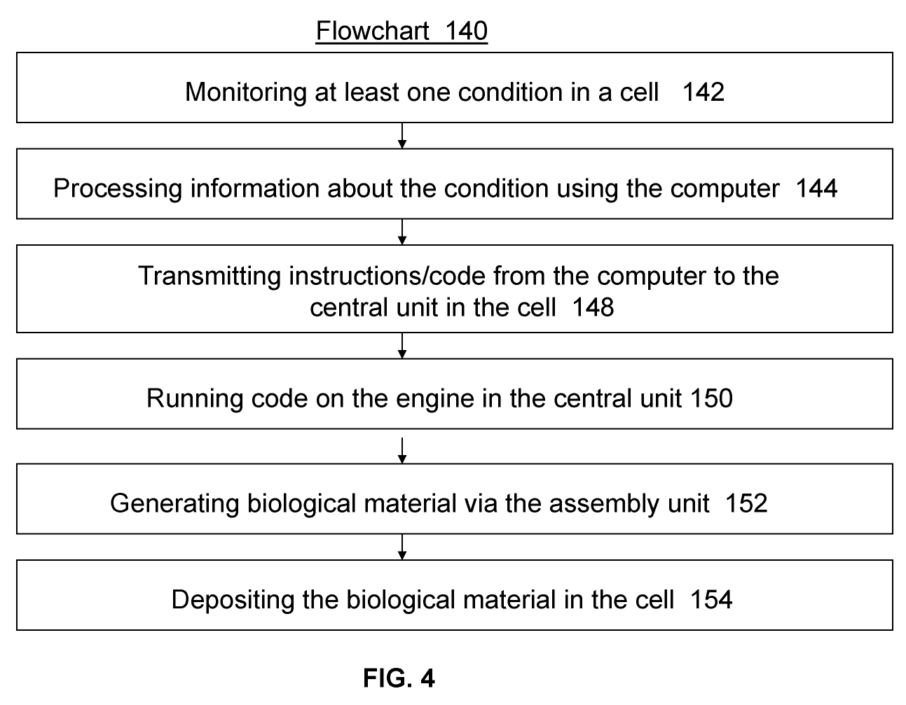

Referring to FIG. 4, a process 140 for modulating cell activity is shown. The process 140 includes monitoring 142 at least one condition (e.g., pH, hypotonicity, temperature) in a cell. The information about the cell is processed 144 by the computer, and a transmitter 6 transmits 148 the information to a central unit 10. The monitoring step can include monitoring a condition visually, such as by monitoring a condition of the human, or the monitoring step can include measuring conditions within the cell by sensors located on one or more components of the system. The central unit receives the information and sends instructions to the assembly unit in the cell. The central unit can be located in the cell, or the central unit can reside outside the cell (e.g., included in the computer 5). A wireless device, such as a remote control device, located outside the cell can be used to send instructions to the central unit 10 in the cell to direct the synthesis of particular biological material. The assembly unit executes 152 the instructions and generates biological material. The assembly unit then deposits 154 the material into the cell.

Uses. The system and cells featured can be used to treat a disorder, such as a proliferative disorder (e.g., a cancer). For example, to eliminate unwanted cells (e.g., tumor cells) in a human, the computer can be programmed to synthesize a protein that is toxic to a cell, such as an α-sarcin polypeptide. The toxic polypeptide can mimic a polypeptide of any origin, such as of mammalian (e.g., human) origin, bacterial origin, or fungal origin. Alternatively, the computer can be programmed to synthesize a double-stranded RNA (dsRNA), such as a short hairpin RNA, that can downregulate gene expression by hybridizing to an endogenous RNA of the human, effectively shutting down translation of the endogenous RNA by the process of RNA interference. The result is the death of the cell and subsequently, the death of the tumor. In another alternative, the computer can be programmed to synthesize a single-stranded antisense RNA or microRNA, that can downregulate gene expression by hybridizing to an endogenous RNA of the human.

The computer can be programmed to synthesize a dsRNA for the downregulation of any gene, and a computer can be programmed according to the features of the target cell. For example, the computer can be programmed to synthesize a dsRNA that targets a Src gene, such as for treating tumors of the colon. A computer programmed to synthesize a dsRNA that targets the RAS gene can be used to treat tumors of the pancreas, colon or lung, and a computer programmed to synthesize a dsRNA that targets the c-MYC gene can be used to treat a neuroblastoma.

A system can be introduced into the unwanted cell by any method described below. For example, microprojectile technology can be used to propel a system into the cells of a tumor mass.

The systems can be delivered to a human through use of a tissue graft. For example, cells can be cultured in vitro for use in a tissue graft. Before transferring the tissue to a human, the systems are introduced into the cells of the graft, such as through a liposomal carrier, or by an electrical pulse. The systems delivered to the tissue graft can be programmed to synthesize a therapeutic biological component, e.g., a therapeutic nucleic acid or polypeptide. In particular, a therapeutic polypeptide synthesized by the system can be secreted by the cells of the tissue graft, where they are taken up by neighboring cells in need of therapeutic polypeptides. The tissue grafts can be applied to diseased or damaged tissue, e.g., to treat burns or diseased organs, such as diseased heart, liver, or kidney tissue.

A system can replace a cell's nucleus. For example, the nucleus can be removed, such as by micromanipulation, and a system can then be injected into the cell. The system is programmed with the information needed to synthesize the biological material and to maintain cell growth and survival. For example, adult neural cells can be subjected to an exchange of a nucleus for a system. The system is programmed with information regarding proteins required for neural cell survival and neurite outgrowth. Neural cells carrying a system can be transplanted into patients having a neurological disorder, such as due to genetic disposition or trauma, to replace nerve function. For example, the cells can be transplanted into or near the spinal cord of paraplegic patients to restore function to the central nervous system, and consequently to improve or restore mobility.

A system can be used to treat a human having a variety of different disorders. As discussed above, a human having a cancer can be treated with a system. For example, the human can have colon cancer, breast cancer, pancreatic cancer, lung cancer, liver cancer, gall bladder cancer, endometrial cancer, a glioblastoma, a squamous cell carcinoma, ovarian cancer, prostate cancer, Ewing Sarcoma, myxoid liposarcoma, leukemia, an adenocarcinoma, and the like.

A system can be also be used to treat a human who experiences acute or chronic pain, or an autoimmune disorder. Exemplary autoimmune disorders include, but are not limited to, rheumatoid arthritis, systemic lupus erythematosus, Sjögren's syndrome, scleroderma, mixed connective tissue disease, dermatomyositis, polymyositis, Reiter's syndrome or Behcet's disease, type I (Insulin dependent), type II diabetes mellitus, Hashimoto's thyroiditis, Graves' Disease, multiple sclerosis, myasthenia gravis, encephalomyelitis, phemphigus vulgaris, phemphigus vegetans, phemphigus foliaceus, Senear-Usher syndrome, Brazilian phemphigus, psoriasis (e.g., psoriasis vulgaris), atopic dermatitis, inflammatory bowel disease (e.g., ulcerative colitis or Crohn's Disease), a disorder resulting from an organ, tissue, or cell transplant (e.g., a bone marrow transplant), such as acute or chronic GVHD, or aplastic anemia, endogenous uveitis, nephrotic syndrome, primary biliary cirrhosis, lichen planus, pyoderma gangrenosum, alopecia areata, a Bullous disorder, chronic viral active hepatitis, auto immune chronic active hepatitis, acquired immune deficiency syndrome (AIDS), and the like.

A system can be used to treat a human infected with a pathogen, e.g., a virus, bacteria, or fungus. For example, the human can have a virus, such as a hepatitis virus (e.g., Hepatitis A, B, C, D, E, F, G H), respiratory syncitial virus, Herpes simplex virus, cytomegalovirus, Epstein Barr Virus, Kaposi's Sarcoma-associated Herpes Virus, JC Virus, rhinovirus, myxovirus, coronavirus, West Nile Virus, St. Louis Encephalitis flavivirus, Tick-borne encephalitis virus, Murray Valley encephalitis flavivirus, Simian Virus 40, Human T Cell Lymphotropic Virus, Moloney-Murine Leukemia Virus, encephalomyocarditis virus, measles virus, Vericella zoster virus, yellow fever virus, adenovirus, poliovirus, poxvirus, and the like.

The human can be infected with a bacteria, such as Mycobacterium ulcerans, Mycobacterium tuberculosis, Mycobacterium leprae, Staphylococcus aureus, Streptococcus pneumoniae, Streptococcus pyogenes, Chlamydia pneumoniae, Mycoplasma pneumoniae, and the like.

A human can be treated with other pharmaceutical compositions, or other therapy regimens, in addition to treatment with a system. The methods can also be used therapeutically or prophylactically.

A cell containing a system can generate large amounts of a protein that are secreted and harvested for use as therapeutic agents. For this purpose, the system in the cell is programmed to synthesize a particular polypeptide of interest. For example, the system can be programmed to synthesize large quantities of insulin for packaging and marketing for the treatment of diabetes or human growth hormone for treatment of dwarfism in children. The system can be programmed to synthesize any variation of a polypeptide, including variants discovered to have greater efficacy or fewer side effects than naturally occurring polypeptides.

The compositions and methods provided may also be used, e.g., as a research tool, to examine the function of various proteins and genes in vitro in cultured or preserved dermal tissues and in animals. The system can be applied to examine the function of any gene.

Delivery of a System to a Cell.

A system can be introduced into a cell by any method, including any method traditionally used to introduce nucleic acids into cells. For example, a system can be introduced into a cell by microinjection, electroporation, by liposomes, or by microprojectile technology.

A system can be delivered to a cell as a component of a membranous molecular assembly, e.g., a liposome or a micelle. As used herein, the term “liposome” refers to a vesicle composed of amphiphilic lipids arranged in at least one bilayer, e.g., one bilayer or a plurality of bilayers. Liposomes include unilamellar and multilamellar vesicles that have a membrane formed from a lipophilic material and an aqueous interior. The aqueous portion contains the system. The lipophilic material isolates the aqueous interior from an aqueous exterior. Liposomes are generally useful for the transfer and delivery of active ingredients (e.g., a system) to the site of action (e.g., to the interior of a cell). Because the liposomal membrane is structurally similar to biological membranes, when liposomes are applied to a tissue, the liposomal bilayer fuses with the bilayer of the cellular membranes. As the merging of the liposome and cell progresses, the internal aqueous contents that include the system are delivered into the cell where the system can synthesize biological components. In some cases the liposomes are also specifically targeted, e.g., to direct the system to a particular cell type (see methods of targeting below).

A liposome containing a system can be prepared by a variety of methods. In one example, the lipid component of a liposome is dissolved in a detergent so that micelles are formed with the lipid component. For example, the lipid component can be an amphipathic cationic lipid or lipid conjugate. The detergent can have a high critical micelle concentration and may be nonionic. Exemplary detergents include cholate, CHAPS, octylglucoside, deoxycholate, and lauroyl sarcosine. Systems are then added to the micelles that include the lipid component. The system can be coated with an anionic material such that the cationic groups on the lipid interact with the system and condense around the system to form a liposome. After condensation, the detergent is removed, e.g., by dialysis, to yield a liposomal preparation containing the system.

If necessary a carrier compound that assists in condensation can be added during the condensation reaction, e.g., by controlled addition. For example, the carrier compound can be a polymer, such as spermine or spermidine. pH can also adjusted to favor condensation.

One maj or type of liposomal composition includes phospholipids other than naturally-derived phosphatidylcholine. Neutral liposome compositions, for example, can be formed from dimyristoyl phosphatidylcholine (DMPC) or dipalmitoyl phosphatidylcholine (DPPC). Anionic liposome compositions generally are formed from dimyristoyl phosphatidylglycerol, while anionic fusogenic liposomes are formed primarily from dioleoyl phosphatidylethanolamine (DOPE). Another type of liposomal composition is formed from phosphatidylcholine (PC) such as, for example, soybean PC, and egg PC. Another type is formed from mixtures of phospholipid and/or phosphatidylcholine and/or cholesterol.

Examples of other methods to introduce liposomes into cells in vitro and in vivo include U.S. Pat. Nos. 5,283,185; 5,171,678; WO 94/00569; WO 93/24640; WO 91/16024; Felgner, J. Biol. Chem. 269:2550, 1994; Nabel, Proc. Natl. Acad. Sci. 90:11307, 1993; Nabel, Human Gene Ther. 3:649, 1992; Gershon, Biochem. 32:7143, 1993; and Strauss EMBO J. 11:417, 1992.

One cationic lipid conjugate includes derivatization of the lipid with cholesterol (“DC-Chol”) which has been formulated into liposomes in combination with DOPE (See, Gao, X. and Huang, L., Biochim. Biophys. Res. Commun. 179:280, 1991). Lipopolylysine, made by conjugating polylysine to DOPE, has been reported to be effective for transfection in the presence of serum (Zhou, X. et al., Biochim. Biophys. Acta 1065:8, 1991). For certain cell lines, these liposomes containing conjugated cationic lipids, are said to exhibit lower toxicity and provide more efficient transfection than the DOTMA-containing compositions. Other commercially available cationic lipid products include DMRIE and DMRIE-HP (Vical, La Jolla, Calif.) and Lipofectamine (DOSPA) (Life Technology, Inc., Gaithersburg, Md.).

Liposomal formulations are particularly suited for topical administration, liposomes present several advantages over other formulations. Such advantages include reduced side effects related to high systemic absorption of the administered drug, increased accumulation of the administered drug at the desired target, and the ability to administer a system to skin cells. In some implementations, liposomes are used for delivering a system to epidermal cells and also to enhance the delivery of the systems into dermal tissues, e.g., into skin. For example, the liposomes can be applied topically. Topical delivery of drugs formulated as liposomes to the skin has been documented (see, e.g., Weiner et al., Journal of Drug Targeting, 2:405-410, 1992, and du Plessis et al., Antiviral Research, 18:259-265, 1992; Mannino, R. J. and Fould-Fogerite, S., Biotechniques 6:682-690, 1988; Itani, T. et al. Gene 56:267-276. 1987; Nicolau, C. et al. Meth. Enz. 149:157-176, 1987; Straubinger, R. M. and Papahadjopoulos, D. Meth. Enz. 101:512-527, 1983; Wang, C. Y and Huang, L., Proc. Natl. Acad. Sci. USA 84:7851-7855, 1987).

Non-ionic liposomal systems can also be used to deliver a system to the skin. Non-ionic liposomal formulations include Novasome I (glyceryl dilaurate/cholesterol/polyoxyethylene-10-stearyl ether) and Novasome II (glyceryl distearate/cholesterol/polyoxyethylene-10-stearyl ether). Such formulations containing the systems are useful for treating a dermatological disorder.

Surfactants find wide application in formulations such as emulsions (including microemulsions) and liposomes (see above). Compositions including a system can include a surfactant. In one embodiment, the system is formulated as an emulsion that includes a surfactant.

If the surfactant molecule is not ionized, it is classified as a nonionic surfactant. Nonionic surfactants include nonionic esters such as ethylene glycol esters, propylene glycol esters, glyceryl esters, polyglyceryl esters, sorbitan esters, sucrose esters, and ethoxylated esters. Nonionic alkanolamides and ethers such as fatty alcohol ethoxylates, propoxylated alcohols, and ethoxylated/propoxylated block polymers are also included in this class. The polyoxyethylene surfactants are the most popular members of the nonionic surfactant class.

If the surfactant molecule carries a negative charge when it is dissolved or dispersed in water, the surfactant is classified as anionic. Anionic surfactants include carboxylates such as soaps, acyl lactylates, acyl amides of amino acids, esters of sulfuric acid such as alkyl sulfates and ethoxylated alkyl sulfates, sulfonates such as alkyl benzene sulfonates, acyl isethionates, acyl taurates and sulfosuccinates, and phosphates. The most important members of the anionic surfactant class are the alkyl sulfates and the soaps.

If the surfactant molecule carries a positive charge when it is dissolved or dispersed in water, the surfactant is classified as cationic. Cationic surfactants include quaternary ammonium salts and ethoxylated amines. The quaternary ammonium salts are the most used members of this class.

If the surfactant molecule has the ability to carry either a positive or negative charge, the surfactant is classified as amphoteric. Amphoteric surfactants include acrylic acid derivatives, substituted alkylamides, N-alkylbetaines and phosphatides.

A system can be delivered to a cell as a micellar formulation. In micelles amphipathic molecules are arranged in a spherical structure such that all the hydrophobic portions of the molecules are directed inward, leaving the hydrophilic portions in contact with the surrounding aqueous phase. The converse arrangement exists if the environment is hydrophobic.

A mixed micellar formulation suitable for delivery through transdermal membranes may be prepared by combining a system with an alkali metal C8 to C22 alkyl sulphate, and micelle forming compounds. Exemplary micelle forming compounds include lecithin, hyaluronic acid, pharmaceutically acceptable salts of hyaluronic acid, glycolic acid, lactic acid, chamomile extract, cucumber extract, oleic acid, linoleic acid, linolenic acid, monoolein, monooleates, monolaurates, borage oil, evening of primrose oil, menthol, trihydroxy oxo cholanyl glycine and pharmaceutically acceptable salts thereof, glycerin, polyglycerin, lysine, polylysine, triolein, polyoxyethylene ethers and analogues thereof, polidocanol alkyl ethers and analogues thereof, chenodeoxycholate, deoxycholate, and mixtures thereof. The micelle forming compounds may be added at the same time or after addition of the alkali metal alkyl sulphate. Mixed micelles will form with substantially any kind of mixing of the ingredients but vigorous mixing is preferred in order to provide smaller size micelles.

In one method a first micellar composition is prepared which contains the system and at least the alkali metal alkyl sulphate. The first micellar composition is then mixed with at least three micelle forming compounds to form a mixed micellar composition. In another method, the micellar composition is prepared by mixing the composition containing the system, the alkali metal alkyl sulphate and at least one of the micelle forming compounds, followed by addition of the remaining micelle forming compounds, with vigorous mixing.

Phenol and/or m-cresol may be added to the mixed micellar composition to stabilize the formulation and protect against bacterial growth. Alternatively, phenol and/or m-cresol may be added with the micelle forming ingredients. An isotonic agent such as glycerin may also be added after formation of the mixed micellar composition.

The specific concentrations of the essential ingredients can be determined by relatively straightforward experimentation.

In another embodiment, a system may be incorporated into a particle, e.g., a microparticle. Microparticles can be produced by spray-drying, but may also be produced by other methods including lyophilization, evaporation, fluid bed drying, vacuum drying, or a combination of these techniques.

Polymeric particles, e.g., polymeric in microparticles, can be used as a sustained-release reservoir of systems that are taken up by cells only released from the microparticle through biodegradation. The polymeric particles in this embodiment should therefore be large enough to preclude phagocytosis (e.g., larger than 10 μm and preferably larger than 20 μm). Such particles can be produced by the same methods to make smaller particles, but with less vigorous mixing of the first and second emulsions. That is to say, a lower homogenization speed, vortex mixing speed, or sonication setting can be used to obtain particles having a diameter around 100 μm rather than 10 am. The time of mixing also can be altered.

Larger microparticles can be formulated as a suspension, a powder, or an implantable solid, to be delivered by intramuscular, subcutaneous, intradermal, intravenous, or intraperitoneal injection; via inhalation (intranasal or intrapulmonary); orally; or by implantation. These particles are useful for delivery of a system when slow release over a relatively long term is desired. The rate of degradation, and consequently of release, varies with the polymeric formulation.

Microparticles preferably include pores, voids, hollows, defects or other interstitial spaces that allow the fluid suspension medium to freely permeate or perfuse the particulate boundary. For example, the perforated microstructures can be used to form hollow, porous spray dried microspheres.

Polymeric particles containing the systems can be made using a double emulsion technique, for instance. First, the polymer is dissolved in an organic solvent. A preferred polymer is polylactic-co-glycolic acid (PLGA), with a lactic/glycolic acid weight ratio of 65:35, 50:50, or 75:25. Next, systems in aqueous solution are added to the polymer solution and the two are mixed to form a first emulsion. The solutions can be mixed by vortexing or shaking, and in a preferred method, the mixture can be sonicated. Most preferable is any method by which the system receives the least amount of damage while still allowing the formation of an appropriate emulsion. For example, a Vibra-cell model VC-250 sonicator is useful for making polymeric particles.

Targeting.

In some embodiments, the system is targeted to a particular cell. For example, a liposome or particle or other structure that includes a system can also include a targeting moiety that recognizes a specific molecule on a target cell. The targeting moiety can be a molecule with a specific affinity for a target cell. Targeting moieties can include antibodies directed against a protein found on the surface of a target cell, or the ligand or a receptor-binding portion of a ligand for a receptor found on the surface of a target cell. For example, the targeting moiety can recognize a cancer-specific antigen (e.g., CA15-3, CA19-9, CEA, or HER2/neu) or a viral antigen, thus delivering the system to a cancer cell or a virus-infected cell. Exemplary targeting moieties include antibodies (such as IgM, IgG IgA, IgD, and the like, or a functional portion thereof), or ligands for cell surface receptors.

Route of Delivery.

A composition that includes a system can be delivered to a human subject by a variety of routes. Exemplary routes include intravenous, topical, nasal, pulmonary, and ocular.

The systems can be incorporated into pharmaceutical compositions suitable for administration. Such compositions typically include at least one system and a pharmaceutically acceptable carrier. As used herein the language “pharmaceutically acceptable carrier” is intended to include any and all solvents, dispersion media, coatings, antibacterial and antifungal agents, isotonic and absorption delaying agents, and the like, compatible with pharmaceutical administration. The use of such media and agents for pharmaceutically active substances is well known in the art. Except insofar as any conventional media or agent is incompatible with the system, use thereof in the compositions is contemplated.

Pharmaceutical compositions featured in may be administered in a number of ways depending upon whether local or systemic treatment is desired and upon the area to be treated. Administration may be topical (including ophthalmic, intranasal, transdermal), oral or parenteral. Parenteral administration includes intravenous drip, subcutaneous, intraperitoneal or intramuscular injection, or intrathecal or intraventricular administration.

The route and site of administration may be chosen to enhance targeting. For example, to target muscle cells, intramuscular injection into the muscles of interest would be a logical choice. Lung cells might be targeted by administering the composition containing the system in aerosol form. The vascular endothelial cells could be targeted by coating a balloon catheter with a composition including the systems and mechanically introducing the composition.

Formulations for topical administration may include transdermal patches, ointments, lotions, creams, gels, drops, suppositories, sprays, liquids and powders. Conventional pharmaceutical carriers, aqueous, powder or oily bases, thickeners and the like may be necessary or desirable.

Compositions for oral administration include powders or granules, suspensions or solutions in water, syrups, elixirs or non-aqueous media, tablets, capsules, lozenges, or troches. In the case of tablets, carriers that can be used include lactose, sodium citrate and salts of phosphoric acid. Various disintegrants such as starch, and lubricating agents such as magnesium stearate, sodium lauryl sulfate and talc, are commonly used in tablets. For oral administration in capsule form, useful diluents are lactose and high molecular weight polyethylene glycols. When aqueous suspensions are required for oral use, the nucleic acid compositions can be combined with emulsifying and suspending agents. If desired, certain sweetening and/or flavoring agents can be added.

Compositions for intrathecal or intraventricular administration may include sterile aqueous solutions which may also contain buffers, diluents and other suitable additives.

Formulations for parenteral administration may include sterile aqueous solutions which may also contain buffers, diluents and other suitable additives. Intraventricular injection may be facilitated by an intraventricular catheter, for example, attached to a reservoir. For intravenous use, the total concentration of solutes should be controlled to render the preparation isotonic.

For ocular administration, ointments or droppable liquids may be delivered by ocular delivery systems known to the art such as applicators or eye droppers. Such compositions can include mucomimetics such as hyaluronic acid, chondroitin sulfate, hydroxypropyl methylcellulose or poly(vinyl alcohol), preservatives such as sorbic acid, EDTA or benzylchronium chloride, and the usual quantities of diluents and/or carriers.

Iontophoresis (transfer of ionic solutes through biological membranes under the influence of an electric field) (Lee et al., Critical Reviews in Therapeutic Drug Carrier Systems, 1991, p. 163), phonophoresis or sonophoresis (use of ultrasound to enhance the absorption of various therapeutic agents across biological membranes, notably the skin and the cornea) (Lee et al., Critical Reviews in Therapeutic Drug Carrier Systems, 1991, p. 166), and optimization of vehicle characteristics relative to dose position and retention at the site of administration (Lee et al., Critical Reviews in Therapeutic Drug Carrier Systems, 1991, p. 168) may be useful methods for enhancing the transport of topically applied compositions across skin and mucosal sites.

Other embodiments are within the scope of the following claims.

1-84. (canceled)

85. A system comprising:
a database storing records comprising records or fields according to the type of database, with the records or fields comprising information of a human's genetic code, altered to replace deleterious code information, with benign or beneficial information;
an assembly unit electrically coupled to a computer system and configured to synthesize biological material based on instructions for assembling the biological material to be synthesized, with the assembly unit further including an input channel and an output channel; and
a computer programmed according to the altered information to control the assembly unit, the computer receives information from the database to construct instructions for synthesizing one type of biological material to support cell function to generate the human's genetic code altered with the benign or beneficial information.

86. The system of claim 85, further comprising an insertion unit that receives synthesized material from the assembly unit for insertion into a cell.

87. The system of claim 85, further comprising a repository unit that stores stock of nucleotides and amino acids.

88. The system of claim 87, wherein the repository unit comprises one or more different types of monomeric biological components.

89. The system of claim 85, wherein the synthesizer unit synthesizes nucleic acids and/or polypeptides.

90. The system of claim 85, further comprising a separate device for a user to communicate wirelessly with the computer, and the computer is further configured to be:
programmed and re-programmed by the separate user device located outside of the cell.

91. The system of claim 85, wherein the computer comprises:
a processor; and
a memory unit, with the process executing software that generates the instructions for assemble based on the information from the database.

92. The system of claim 85, wherein the database further comprises one or more of DNA sequence, RNA sequence and polypeptide sequence information.

93. The system of claim 85, wherein the monomeric biological components include one or more of a nucleotide, amino acid, and tRNA with each tRNA attached to an amino acid.

94. The system of claim 85, wherein the assembly unit comprises one or more of a polymerase or a ribosome that harvests biological material precursor materials directly from cell cytoplasm and through the input channel and releases synthesized biological material into the cell through the output channel.

95. The system of claim 94 wherein the biological material is a nucleic acid or polypeptide.

96. The system of claim 94 wherein the biological material is an RNA.

97. The system of claim 84 wherein the system is coated with a biocompatible material.

98. The system of claim 84 wherein the system further comprises:
a central unit responsive to execution of the instructions to control an assembly unit;
sensors that monitor conditions within the cell; and wherein the central unit receives information from the sensors and sends instructions to the assembly unit that is further electrically connected to the central unit, with the assembly unit being configured to synthesize the biological material based on the instructions executed by the computer.

99. The system of claim 98 wherein the central unit comprises:
an antenna, and the central unit receives the instructions via a wireless connection.

100. The system of claim 98 wherein the computer is separate from the central unit and the assembly unit and the central unit are outside the cell.

101. The system of claim 98 wherein the computer is separate from the central unit and the assembly unit and the central unit are inside the cell.

102. The system of claim 85, further comprising a separate device for a user to communicate wirelessly with the computer.

103. A method comprising:
treating a human comprising
administering a cell comprising a system;
synthesizing biological material that is administered into the cell, wherein the biological material is synthesized by a system comprising:
a database storing records comprising records or fields according to the type of database, with the records or fields comprising information of a human's genetic code, altered to replace deleterious code information, with benign or beneficial information;
an assembly unit electrically coupled to a computer system and configured to synthesize biological material based on instructions for assembling the biological material to be synthesized, with the assembly unit further including an input channel and an output channel; and
a computer programmed according to the altered information to control the assembly unit, the computer receives information from the database to construct instructions for synthesizing one type of biological material to support cell function to generate the human's genetic code altered with the benign or beneficial information.

104. The method of claim 103 wherein synthesizing the biological material is initiated by a signal from a user operating a device separated from the system, wherein the user uses the device to send a signal to a receiver located in the computer of the system.

105. The method of claim 103 wherein the system comprises a central unit that is responsive to instructions to control the assembly unit.

106. The method of claim 103 further comprising:
administering the cell by tissue graft.

107. The method of claim 103 wherein the assembly unit includes one or more of a polymerase or a ribosome or a robot that mimics the activity of a polymerase or a ribosome.
